SEQUENCE LISTING (4 sequences)

SEQ ID NO: 1 - protein, 507 aa, Parabacteroides distasonis
  UNSURE 215
MKNIWKNVLGVALIAAISSGAAIGTSTYLMNKNQRPAELASGVENTFKQPYRLTNYGTVAAENIDFTTAAESAIHGVVHIKATANAQASNGDGGQQYMDPFEYFFGFGGRGGFQRPQQQPRVGAGSGVIISTDGYIITNNHVIDGADELEVTLNDNRKFPAKIIGADPTTDIALIKIEATDLPTIPFGDSEKLKVGEWVLAVGNPFNLTSTVTAXIVSAKSRGNIGAGGKDRSKIESFIQTDAAVNPGNSGGALVNTKGELVGINTAIYSETGNFAGYSFAVPISIAGKVANDLKQFGTVQRAVLGVLIQDPQYVPDAEKEKVKVFEGAYVGGFAERSSAKEAGIEKGDVIVAVNGVKIKSSSALQEQISKYRPGDKVELTINRNGSTKKFTVELRNAQGSTKVVKGGDSAEVMGAAFKALNDEQKRKLGVSYGIEVTGLTSGKLKDAGIKKGFIIMIVNNQKISAPEDLEKIVESILQGRTEDQGLFIKGFYPNGRTKYYAIDLAE

SEQ ID NO: 2 - DNA, 1521 bp, Parabacteroides distasonis
  source 1..1521: mol_type = genomic DNA
atgaaaaatatctggaagaatgtgcttggtgttgctctgatagcagccattagttctggtgccgcgatcggaacgagtacttacctgatgaataagaatcaacgtccggcggagttggcctccggagttgagaataccttcaagcaaccgtaccgattgacgaattatggaaccgtggccgccgagaatatcgatttcactacggccgccgagagcgccatccacggggttgtccatatcaaggccacggctaatgcgcaagcctctaacggggacggcggtcagcagtatatggacccgttcgagtatttcttcggcttcggtggccgtggagggttccaacgtccgcaacagcaacctcgtgtgggcgccggctcgggtgtcattatctctacggatggttatatcatcacgaacaaccacgtgatcgacggagcagatgagctggaggttaccttgaacgataaccggaaattcccggcaaagattataggggctgatccgacaacagatatcgccttgatcaagatcgaggcaaccgacttacctacgatcccctttggtgattctgagaagttgaaagtaggtgagtgggtgttggcggtaggtaacccgtttaacttgacctctaccgttacggcaggkatcgtgagcgctaagagccgtggtaacatcggcgccggtggcaaggatcgtagcaagatcgagtcgtttatacagacagacgccgccgtgaacccgggtaacagtggtggggcgctagtgaacacgaaaggcgagttagtcggtatcaataccgctatctactccgagaccggtaacttcgccggatactctttcgccgtgcctatcagcatcgccggtaaggtggctaacgacttgaagcaatttggaaccgtgcagcgtgctgtattaggcgtattgatacaagacccgcaatacgtgcccgacgctgagaaagagaaagtgaaagtattcgagggcgcttacgtaggtggtttcgccgagcgcagctccgctaaggaagccggaatcgagaagggtgacgtgattgtagctgtgaacggcgtgaagatcaaatcgtctagcgctttgcaagagcagatcagcaaataccgtccgggcgacaaggtagagttgacgatcaaccgcaacggtagcacgaagaaattcacggtagaactccgtaatgcgcaaggtagcaccaaggtcgtgaagggtggcgacagcgcagaggttatgggcgcagcgttcaaggctttgaacgacgagcagaaacgtaagttaggtgtaagttatggtatcgaggtcaccggtctgaccagcggcaaactgaaagacgccggtataaagaaaggcttcattatcatgattgtaaacaaccagaagatctctgctccggaagacttggaaaagattgtggaaagcatacttcaaggacgtacggaagatcaaggcctcttcattaaaggcttctacccgaacggacgtacgaagtactatgcgatagatcttgccgaa

SEQ ID NO: 3 - protein, 484 aa, Parabacteroides distasonis
  UNSURE 192
MTSTYLMNKNQRPAELASGVENTFKQPYRLTNYGTVAAENIDFTTAAESAIHGVVHIKATANAQASNGDGGQQYMDPFEYFFGFGGRGGFQRPQQQPRVGAGSGVIISTDGYIITNNHVIDGADELEVTLNDNRKFPAKIIGADPTTDIALIKIEATDLPTIPFGDSEKLKVGEWVLAVGNPFNLTSTVTAXIVSAKSRGNIGAGGKDRSKIESFIQTDAAVNPGNSGGALVNTKGELVGINTAIYSETGNFAGYSFAVPISIAGKVANDLKQFGTVQRAVLGVLIQDPQYVPDAEKEKVKVFEGAYVGGFAERSSAKEAGIEKGDVIVAVNGVKIKSSSALQEQISKYRPGDKVELTINRNGSTKKFTVELRNAQGSTKVVKGGDSAEVMGAAFKALNDEQKRKLGVSYGIEVTGLTSGKLKDAGIKKGFIIMIVNNQKISAPEDLEKIVESILQGRTEDQGLFIKGFYPNGRTKYYAIDLAE

SEQ ID NO: 4 - DNA, 1452 bp, Parabacteroides distasonis
  source 1..1452: mol_type = genomic DNA
atgacgagtacttacctgatgaataagaatcaacgtccggcggagttggcctccggagttgagaataccttcaagcaaccgtaccgattgacgaattatggaaccgtggccgccgagaatatcgatttcactacggccgccgagagcgccatccacggggttgtccatatcaaggccacggctaatgcgcaagcctctaacggggacggcggtcagcagtatatggacccgttcgagtatttcttcggcttcggtggccgtggagggttccaacgtccgcaacagcaacctcgtgtgggcgccggctcgggtgtcattatctctacggatggttatatcatcacgaacaaccacgtgatcgacggagcagatgagctggaggttaccttgaacgataaccggaaattcccggcaaagattataggggctgatccgacaacagatatcgccttgatcaagatcgaggcaaccgacttacctacgatcccctttggtgattctgagaagttgaaagtaggtgagtgggtgttggcggtaggtaacccgtttaacttgacctctaccgttacggcaggkatcgtgagcgctaagagccgtggtaacatcggcgccggtggcaaggatcgtagcaagatcgagtcgtttatacagacagacgccgccgtgaacccgggtaacagtggtggggcgctagtgaacacgaaaggcgagttagtcggtatcaataccgctatctactccgagaccggtaacttcgccggatactctttcgccgtgcctatcagcatcgccggtaaggtggctaacgacttgaagcaatttggaaccgtgcagcgtgctgtattaggcgtattgatacaagacccgcaatacgtgcccgacgctgagaaagagaaagtgaaagtattcgagggcgcttacgtaggtggtttcgccgagcgcagctccgctaaggaagccggaatcgagaagggtgacgtgattgtagctgtgaacggcgtgaagatcaaatcgtctagcgctttgcaagagcagatcagcaaataccgtccgggcgacaaggtagagttgacgatcaaccgcaacggtagcacgaagaaattcacggtagaactccgtaatgcgcaaggtagcaccaaggtcgtgaagggtggcgacagcgcagaggttatgggcgcagcgttcaaggctttgaacgacgagcagaaacgtaagttaggtgtaagttatggtatcgaggtcaccggtctgaccagcggcaaactgaaagacgccggtataaagaaaggcttcattatcatgattgtaaacaaccagaagatctctgctccggaagacttggaaaagattgtggaaagcatacttcaaggacgtacggaagatcaaggcctcttcattaaaggcttctacccgaacggacgtacgaagtactatgcgatagatcttgccgaa

(19) United States
(12) Patent Application Publication
(10) Pub. No.: US 2025/0375507 A1
(43) Pub. Date: Dec. 11, 2025

(54) Protease Formulation for Treatment of Toxins

(71) Applicants: Queen's University at Kingston, Kingston (CA); Kingston Health Sciences Centre, Kingston (CA)

(72) Inventors: Prameet Sheth, Kingston (CA); Shu-Mei He, Kingston (CA); Curtis Noordhof

(21) Appl. No.: 18/874,304

(22) PCT Filed: Jun. 28, 2023
(86) PCT No.: PCT/CA2023/050889

§ 371 Date: Dec. 12, 2024

Related U.S. Application Data
(60) Provisional application No. 63/356,443, filed on Jun. 28, 2022.

(51) Int. Cl. A61K 38/48; A61P 31/04; C12N 9/52

(52) U.S. Cl. CPC A61K 38/482; A61P 31/04; C12N 9/52; C12Y 304/21107

(57) ABSTRACT

A method for treatment of one or more toxin(s) in a subject, that includes administering to the subject a therapeutically effective amount of a serine protease that enzymatically cleaves certain toxins. Also, a method for preventing disease progression in a subject infected by Clostridioides difficile (CD), diarrhea, or infectious colitis, which includes administering to the subject a therapeutically effective amount of the protease. The protease retains its activity up to 65° C. Treatment with the protease in mice infected with CD conferred a 10-fold survival benefit compared to mice infected with CD and untreated.

### RELATED APPLICATION

This application claims the benefit of the filing date of U.S. Application No.: 63/356,443, filed on Jun. 28, 2022, the contents of which are incorporated herein by reference in their entirety.

### FIELD The field of the invention is protease treatment to degrade a toxin.

### BACKGROUND

Clostridioides (formerly Clostridium) difficile (CD) is the leading cause of hospital-associated diarrhea. Subjects with CD infection (CDI) can experience a range of symptoms from mild diarrhea to pseudomembranous colitis, toxic megacolon, and even death. The pathogenesis of CD is toxin-mediated. Toxin A (TcdA) and toxin B (TcdB) catalyze glycosylation and inactivation of human Rho-GTPases. Inactivation of small regulatory proteins leads to disorganization of the cell cytoskeleton, and to cell-death. Rho proteins are important in the maintenance of epithelium tight junctions. Toxins disrupt the epithelial barrier, leading to pseudomembranous colitis.

Currently, CD treatment involves use of antibiotics, which have a 20-25% failure rate. In subjects that fail antibiotics repeatedly, the Food and Drug Association of the USA and Health Canada have authorized the use of human fecal transplants, which recolonize the human gut and in many cases resolve the CD. However, fecal transplants require healthy donors, intensive screening of healthy donors for infectious diseases, and are labour intensive.

CD is responsible for over 500,000 infections and >30,000 deaths annually in North America alone. It results in a range of symptoms from mild diarrhea to pseudomembranous colitis, toxic megacolon, colonic perforation and death. CD infection (CDI) results in major challenges for infection control as the CD bacteria readily form spores that are environmentally stable. Risk factors for the development of CDI include antibiotic use, advanced age (>65 years) and exposure to healthcare facility including long-term care. The widespread use of antibiotics, and the increased cohorting of older adults in assisted living facilities has contributed to major CD outbreaks and an increase in prevalence and severity of CDI in last two decades.

There is a need for treatment of CD that has a low failure rate and that is not labour intensive to provide.

### SUMMARY

In one aspect, the invention provides a method for treatment of one or more toxin(s) in a subject, the method comprising administering to the subject a therapeutically effective amount of a protein that comprises HTRA-NS protein (SEQ ID NO: 3). In one aspect, the invention provides a method for treatment of one or more toxin(s) in a subject, the method comprising administering to the subject a therapeutically effective amount of HTRA protein (SEQ ID NO: 1) or HTRA-NS protein (SEQ ID NO: 3). In one embodiment, the one or more toxin(s) are from one or more pathogen(s). In one embodiment, the one or more pathogen(s) is Clostridioides difficile. In one embodiment, the subject is being administered antibiotics. In one embodiment of the method, one or more toxin(s) are selected from Toxin Clostridioides difficile A (TcdA), Toxin Clostridioides difficile B (TcdB), or Toxin Clostridioides difficile binary (referred to herein as CDT). In one embodiment, the method of treatment restores gastrointestinal flora to healthy gastrointestinal flora.

In one aspect, the invention provides a method for preventing disease progression in a subject infected by Clostridioides difficile, diarrhea, or infectious colitis, the method comprising administering to the subject a therapeutically effective amount of a protein that comprises HTRA-NS protein (SEQ ID NO: 3). In one embodiment, the protein is HTRA protein (SEQ ID NO: 1) or HTRA-NS protein (SEQ ID NO: 3). In one embodiment, the one or more toxin(s) is from one or more pathogen(s). In one embodiment, the one or more pathogen(s) is Clostridioides difficile. In one embodiment, the subject is being administered antibiotics. In one embodiment, the one or more toxin(s) is selected from TcdA, TcdB, or CDT.

In one aspect, the invention provides a method for the prophylaxis of one or more toxin(s) in a subject, the method comprising administering to the subject a therapeutically effective amount of a protein that comprises HTRA-NS protein (SEQ ID NO: 3). In one embodiment, the protein is HTRA protein (SEQ ID NO: 1) or HTRA-NS protein (SEQ ID NO: 3). In one embodiment, the one or more toxin(s) is from one or more pathogen(s). In one embodiment, the one or more pathogen(s) is Clostridioides difficile. In one embodiment, the subject is being administered antibiotics. In one embodiment, the one or more toxin(s) is selected from TcdA and TcdB. In one embodiment of the aspects described herein, the subject is a mammal. In one embodiment, the mammal is a human. In one embodiment, the mammal is a swine, cattle, horse, or dog.

In one aspect, the invention provides a pharmaceutical preparation comprising a therapeutically effective amount of a protein comprising HTRA-NS protein (SEQ ID NO: 3) and a pharmaceutically acceptable excipient. In one aspect, the invention provides a pharmaceutical preparation comprising a therapeutically effective amount of HTRA protein (SEQ ID NO: 1) or HTRA-NS protein (SEQ ID NO: 3). In one aspect, the invention provides a pharmaceutical preparation comprising a therapeutically effective amount of HTRA protein (SEQ ID NO: 1) or HTRA-NS protein (SEQ ID NO: 3) and a pharmaceutically acceptable excipient. In one embodiment, the pharmaceutical preparation is for treatment of one or more toxin(s) in a subject. In one embodiment, the one or more toxin(s) is from one or more pathogen(s). In one embodiment, the one or more pathogen(s) is Clostridioides difficile. In one embodiment, wherein the one or more toxin(s) are selected from TcdA, TcdB, or CDT. In one embodiment, the preparation is in the form of a tablet, lyophilized preparation, solution, syrup, lozenge, suppository, enema, food additive, capsule, spray, gel, cream, lotion, ointment, or foam. In one aspect, the invention provides a composition comprising a protein comprising HTRA-NS (SEQ ID NO: 3). In one aspect, the invention provides a composition comprising HTRA protein (SEQ ID NO: 1) or HTRA-NS (SEQ ID NO: 3) and a pharmaceutically acceptable excipient. In one embodiment, the composition is for treatment of one or more toxin(s). In one embodiment, the composition further comprises one or more additional therapeutic agents. In one embodiment, the one or more additional therapeutic agent(s) is selected from the group consisting of antivirals, antibiotics, antibacterial agents and antiproteases.

In one aspect, the invention provides a protein comprising HTRA-NS (SEQ ID NO: 3). In one embodiment, the protein is a recombinant protein. In one aspect, the invention provides a nucleic acid encoding the protein of SEQ ID NO: 4. In one aspect, the invention provides a pharmaceutical composition comprising this nucleic acid. In one aspect, the invention provides a composition comprising the protein of SEQ ID NO: 4. In one aspect, the invention provides a method comprising administering this composition. In one embodiment of the method of treatment aspect, the treatment is prophylactic treatment.

In one aspect, the invention provides a protein that is suitable for enzymatically cleaving TcdA, TcdB, or CDT. In one aspect, the invention provides a method of preventing disease recurrence in a subject infected by Clostridioides difficile, diarrhea, or infectious colitis.

### BRIEF DESCRIPTION OF THE DRAWINGS

For a better understanding of the invention and to show more clearly how it may be carried into effect, reference will now be made, by way of example, to the accompanying drawings, wherein:

FIG. 1A shows a bar graph of the impact of HTRA-NS on TcdA by Western Blot (WB) Densitometry analysis.

FIG. 1B shows a bar graph of the impact of HTRA-NS on TcdB by Western Blot Densitometry analysis.

FIG. 2A shows a bar graph of HTRA-NS protecting murine lung fibroblasts from TcdA mediated cytoskeletal rearrangement and cell rounding.

FIG. 2B shows a bar graph percent rounded cells vs. stool from mice at the specified number of days post CD infection.

FIG. 3A shows a line graph of changes in percent body weight in the CD infection mouse model, wherein mice lost 20% of their body weight following CD infection and where oral gavage of mice with HTRA-NS (400 ug) provided protection from significant CD-mediated weight loss.

FIG. 3B shows a column dot plot that demonstrates impact of CD on mouse colon, mice exposed to HTRA experienced no changes in their colon length despite being infected with CD; and mice infected with CD but not treated with HTRA-NS demonstrated significant colonic shortening.

FIG. 3C shows a column dot plot that demonstrates impact of CD on mouse colon length. The results demonstrated that CD control mice had significantly more colonic shortening than other groups (p=0.0001 vs. negative control, p=0.009 vs. HTRA-NS control and p=0.04 vs. CD+HTRA-NS.

FIG. 3D shows images of stool from 3 groups of mice (control group with no CD; CD infected group; and CD infected mice that were treated with HTRA-NS) at 24, 48, and 72 hours of incubation with fibroblasts, which resulted in cytoskeletal rearrangement and cell death. Stool from mice with CD that were treated with HTRA-NS showed no cell rounding of fibroblasts, which indicated that the treated group had cells protected from cytoskeletal rearrangement and cell death. Stool from the treated group was similar to stool from uninfected mice which was incubated on fibroblasts at 24, 48, and 72 hours.

FIG. 4A shows impact on weight of mice gavaged with different concentrations of HTRA-NS (200, 400 and 800 ug) following antibiotic therapy, mice gavaged with HTRA-NS experienced no significant weight loss (200, 400,800 ug lines) compared to control mice that did not receive HTRA-NS (Tris buffer control mouse), demonstrating that HTRA therapy does not result in significant weight loss.

FIG. 4B shows a column bar graph illustrating that unlike when infected with CD (FIG. 3B), HTRA-NS exposure at different concentrations (200, 400 and 800 ug) did not result in significant colonic shortening in mice.

FIG. 5A-E shows a series of line graphs that 16S rRNA sequencing of mouse stool exposed to different concentrations of HTRA-NS (200, 400 and 800 ug) did not result in significant changes to the specified predominating gut bacterial populations compared to controls.

FIG. 6 shows a line graph of relative activity of HTRA-NS vs. temperature.

FIG. 7 shows a plot of percent survival vs. days post infection.

### DETAILED DESCRIPTION OF EMBODIMENTS

The pathophysiology of CD disease is complex and involves the depletion of bacterial communities in the gastrointestinal (GI) tract, which facilitates GI colonization by CD bacteria. Following this colonization, Clostridium difficile toxin A (“TcdA”) and Clostridium difficile toxin B (“TcdB”) are produced, both of which are necessary for the development of CD clinical disease. CD strains that do not synthesize toxin A and toxin B, do not cause human disease. Hypervirulent strains of C. difficile (e.g., NAP1/BI/027) emerged that additionally produce a binary toxin (CDT) (see Stieglitz, F. et al., Front. Microbiol. 12:725612 (2021)).

Toxin A is an enterotoxin and induces inflammation, cytokine release, and fluid secretion leading to diarrhea. Toxin B is a cytotoxin that disrupts cytoskeletal architecture of colonic and epithelial cells, thereby catalyzing glycosylation and inactivation of human Rho-GTPases. Both toxins work synergistically and are capable of inducing cell rounding and cytoskeletal rearrangement at picomolar concentrations in cell culture models. Rho-GTPases modulate intracellular actin dynamics and glycosylation of these toxin molecules, resulting in cytoskeletal changes and cell death.

Treatment of CDI via interventions that restore the disrupted GI microbiota have been explored and have shown efficacy. The use of CD-free human stool, called fecal microbial transplants (FMT), were recently added to Clinical Guidelines published by the Infectious Disease Society of America as a therapeutic option for the treatment of recurrent CDI. However, the wide-spread use of FMTs has been impeded by the difficulty of finding appropriate donors and the variability in performance of stool from different donors. Donor-based differences have been linked to a lack of compatibility of bacterial strains found in the donor and recipient stool (Li, S. S. et al. Science 352, 586-589 (2016)).

A defined microbial community (DMC), which is referred to as MET-1, has been developed (see Martz, S. L. et al. J Gastroenterol 52, 452-465 (2017), Petrof, E. O., et al. Benef Microbes 4, 53-65 (2013), and Petrof, E. O. et al. Microbiome 1, 3 (2013)). MET-1 is a collection of 33-bacteria isolated from stool of a healthy donor. These 33 bacterial organisms were cultured in a controlled laboratory setting. MET-1 has been extensively studied in mouse models of CDI and has been used in humans clinically with >90% efficacy (see Petrof, E. O. et al. Microbiome 1, 3, (2013), Munoz, S. et al. Gut Microbes 7, 353-363, (2016), Grady, N. G., et al. Semin Fetal Neonatal Med 21, 418-423 (2016), Martz, S. L. et al. Sci Rep 5, 16094, (2015), and Carlucci, C. et al. Sci Rep 9, 885, (2019).

According to one aspect of the invention there is provided a method for treating a subject with Clostridium difficile (Clostridioides difficile) infection (CDI), by administering to the subject one or more protein(s) that cause degradation of one or more toxin(s) associated with CDI. According to embodiments, the one or more toxins are selected from TcdA, TcdB, and a combination thereof. In an embodiment, the one or more protein(s) is HTRA (SEQ ID NO: 1). In another embodiment, the one or more protein(s) comprises HTRA-NS (SEQ ID NO: 3). In another embodiment, the one or more protein(s) is HTRA-NS (SEQ ID NO: 3). According to some embodiments, treating a subject by administering a pharmaceutical composition as described herein may prevent CDI recurrence in the subject. According to some embodiments, treating a subject by administering a pharmaceutical composition as described herein may include prophylactic treatment. According to some embodiments, treating a subject by administering a pharmaceutical composition as described herein may at least partially restore normal gastrointestinal (GI) flora in the subject.

Another aspect of the invention provides compositions comprising one or more protein(s) selected from HTRA (SEQ ID NO:1), HTRA-NS (SEQ ID NO:3), and combinations thereof. In some embodiments, the compositions may be pharmaceutical compositions. The pharmaceutical compositions may include one or more additional agent, diluent, carrier, excipient, etc., and/or one or more additional therapeutic agent, suitable for administration to a subject. The subject may be human. In some embodiments the pharmaceutical compositions may be useful in treating a subject with CDI. In some embodiments the pharmaceutical compositions may be useful in degrading one or more toxin(s) associated with CDI. According to embodiments, the one or more toxins are selected from TcdA, TcdB, and a combination thereof. In some embodiments the pharmaceutical compositions may be useful in prophylactic treatment of a subject. In some embodiments the pharmaceutical compositions may be useful in at least partially restoring normal gastrointestinal (GI) flora in a subject.

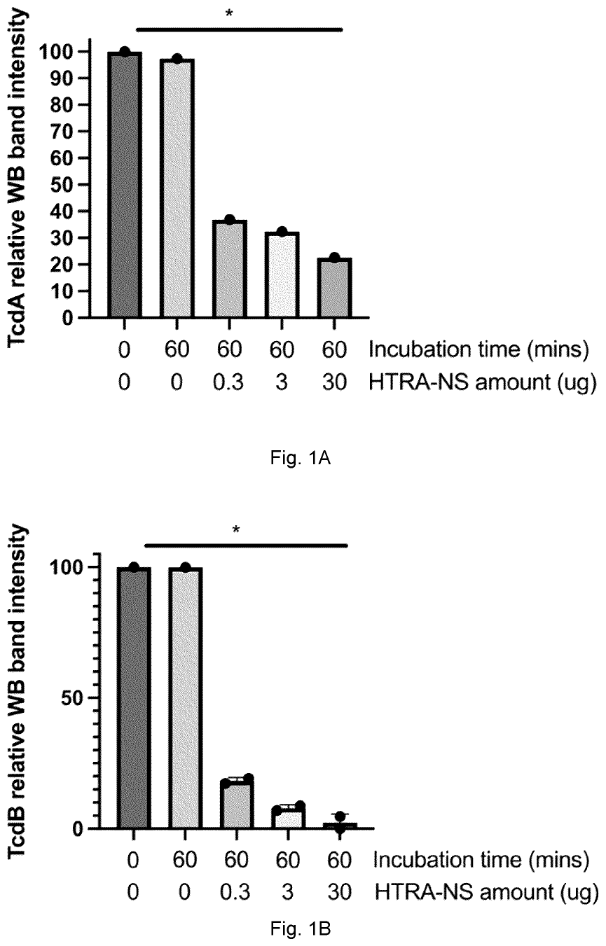

Parabacteriodes distanosis is an anaerobic gram negative bacteria strain. It has a serine protease that has not previously been evaluated for its activity against CD or other toxins. HTRA was obtained from a species of bacteria Parabacteriodes distanosis that can be found in the human gut and is a member of both the bacterial communities of MET-1 and DMC-4. A genetic sequence (SEQ ID NO: 2), which encodes the HTRA protein (SEQ ID NO: 1) was extracted and transferred into Escherichia coli (E. coli). The HTRA protein was expressed in E. coli at high concentrations and HTRA was isolated and purified. HTRA-NS (SEQ ID NO: 3) is a protein that is similar to HTRA in some ways, but differs because the signal peptide has been removed from the gene sequence for HTRA-NS (SEQ ID NO: 4). Removal of the signal peptide allowed for better stability of the resultant protein HTRA-NS (SEQ ID NO: 3) relative to HTRA (SEQ ID NO: 1). A dose-response effect was demonstrated whereby HTRA-NS (SEQ ID NO: 3) degraded both TcdA and TcdB in a concentration dependent manner (see FIGS. 1A-1B). A 70% reduction was demonstrated in cell rounding in cultured fibroblasts which are cells that are very sensitive to CD toxin, in response to toxin A (see details in the Working Examples).

As described herein, experiments were conducted wherein crude stool was collected from mice that were either infected with CD, or infected with CD and treated with HTRA-NS. In mice that were infected with CD and treated with HTRA, the CD toxin activity (TcdA, TcdB and CDT) was neutralized compared to mice infected with CD but not treated with HTRA-NS.

Referring to FIGS. 1A-1B, bar graphs show that pre-incubation of TcdA and TcdB toxins with HTRA protein resulted in degradation of both TcdA and TcdB toxins in an HTRA-NS concentration dependent manner. The highest concentration (i.e., 30 ug) showed a degradation of 80% of TcdA and >95% of TcdB when measured using a Western blot assay. The Western blot bands showed reduced band intensity, a sign that the toxins were not detected, when incubated with increasing concentrations of HTRA-NS (0.3-30 ug).

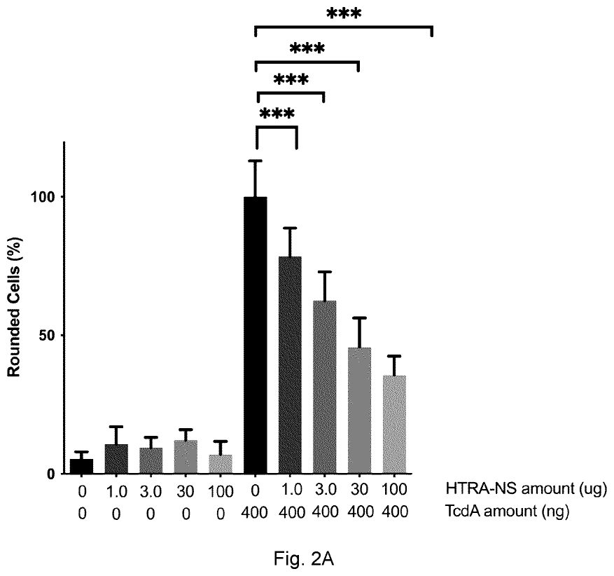

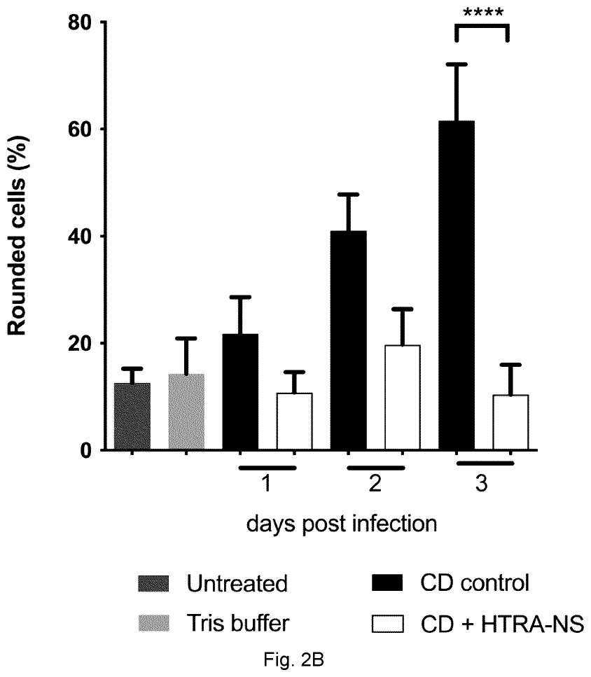

Referring to FIG. 2A, a bar graph shows that purified HTRA-NS protected fibroblasts from TcdA-mediated cytoskeletal rearrangement and cell rounding. The protection increased as the concentration of HTRA was increased, suggesting a dose-response mechanism of protection against TcdA. Cultured 3T3 murine fibroblasts cells exposed to TcdA resulted in significant cytoskeletal rearrangement resulting in cell rounding and death. In contrast, TcdA toxin pre-incubated with concentrations of HTRA-NS (1.0-100 ug) at 37° C. for 1 hour and then added to cultured fibroblasts, resulted in 60% less cell rounding (see last bar). A statistically significant reduction in cell rounding (using a Mann Whitney statistical test) was observed when compared to cells exposed to TcdA alone in an HTRA-NS concentration dependent manner.

Referring to FIG. 2B, a bar graph is shown of percent rounded cells vs. stool from mice at a specified number of days post CD infection. The results showed that stool from CD+HTRA-NS mice rounded less toxin than stool from CD control mice at 1 and 2 days post infection and significantly less than mice of 3 days post infection (p=0.0001).

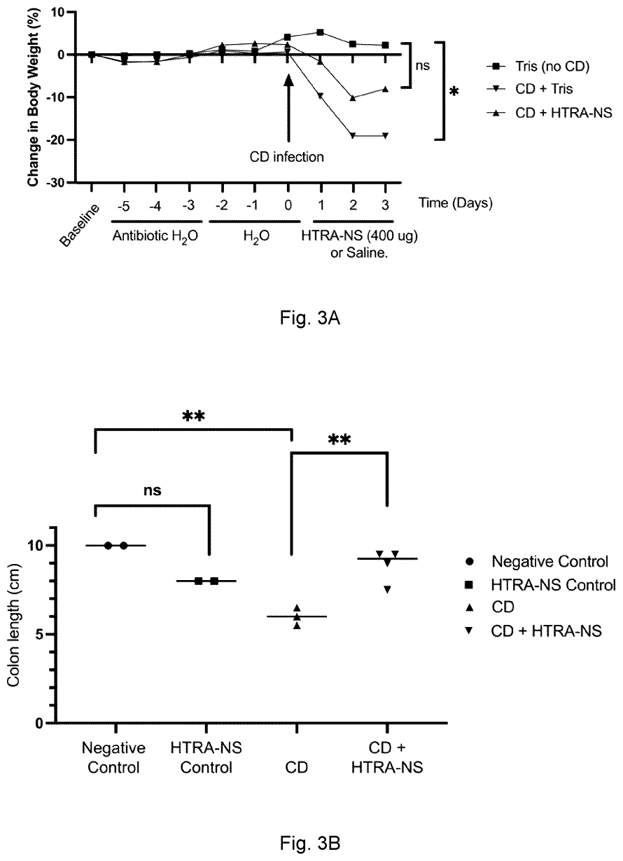

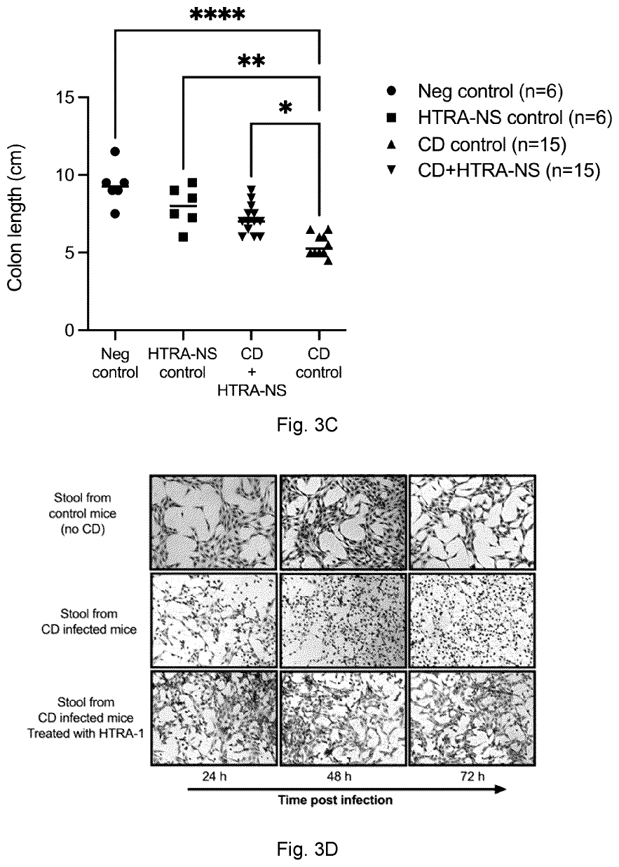

Referring to FIG. 3A, a line graph is shown depicting changes in percent body weight in the CD infection mouse model. Mice lost 20% of their body weight following CD infection and oral gavage of mice with HTRA-NS (400 ug) provided protection from significant CD-mediated weight loss.

Referring to FIG. 3B, a column dot plot is shown that demonstrates impact of CD on mouse colon including colonic shortening. Mice exposed to HTRA-NS experienced no changes in their colon length despite being infected with CD. Mice infected with CD, not treated with HTRA-NS, demonstrated significant colonic shortening. In subsequent in depth studies (see FIG. 3C), which were conducted 3 times with 15 mice in each experiment and included negative controls (2), CD-positive control (n=5), CD+HTRA-NS (n=5) and negative controls (n=2), colon length demonstrated that CD control mice had significantly more colonic shortening than the other groups (i.e, HTRA-NS control, CD+HTRA-NS, and negative controls; p=0.0001 vs Neg control, p=0.009 vs HTRA-NS control and p=0.04 vs CD+HTRA-NS). Total histology scores of distal colonic mucosa revealed that CD+HTRA-NS mice had significantly lower levels of inflammation than CD control mice (p=0.03), however significantly higher than negative control and HTRA control mice (p=0.01).

Referring to FIG. 3D, timepoints for the stool from CD infected mice were 48 and 72 hours of incubation. Stool from mice that were treated with HTRA-NS did not result in cell rounding of fibroblasts and protected cells from cytoskeletal rearrangement and cell death similar to what was observed when stool from CD uninfected mice is incubated on fibroblasts at 48 and 72 hours.

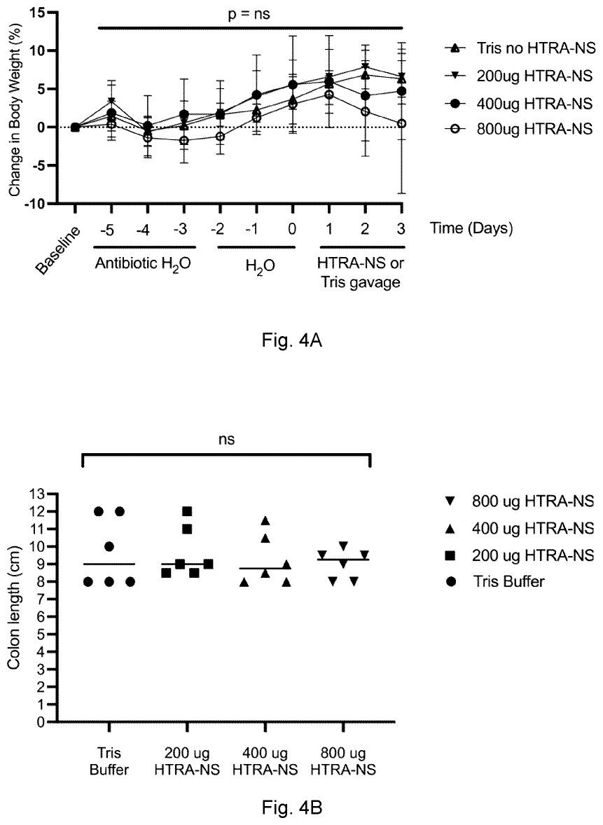

Referring to FIG. 4A, a line graph shows the impact on the weight of mice gavaged with different concentrations of HTRA-NS (200, 400 and 800 ug) following antibiotic therapy. Mice gavaged with HTRA-NS experienced no significant weight loss (200, 400,800 ug lines) compared to control mice that did not receive HTRA-NS (Tris buffer control mouse), demonstrating that HTRA therapy does not result in significant weight loss, a hallmark of colitis in this mouse model.

Referring to FIG. 4B a column bar graph illustrates that unlike when infected with CD (FIG. 3B), HTRA-NS exposure at different concentrations (200, 400 and 800 ug) did not result in significant colonic shortening in mice.

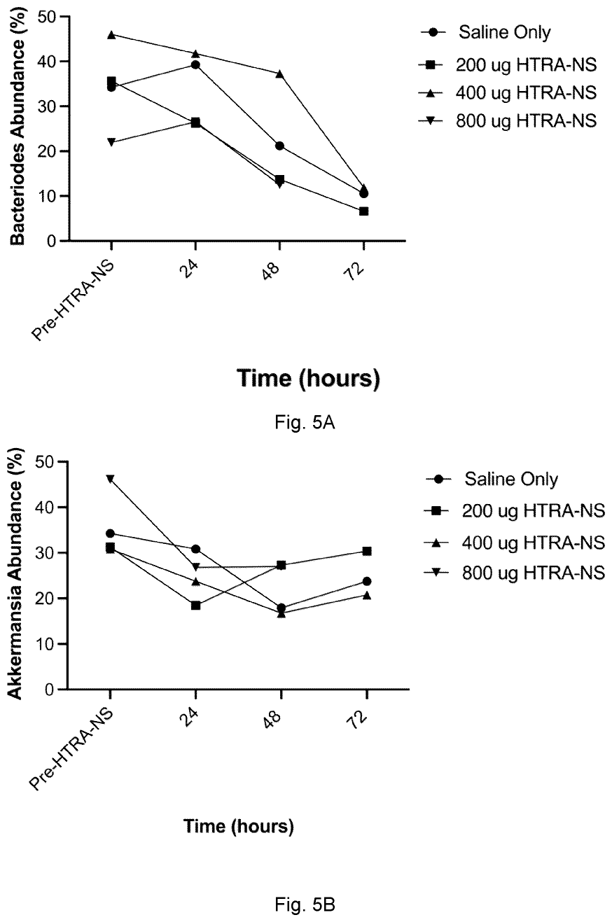

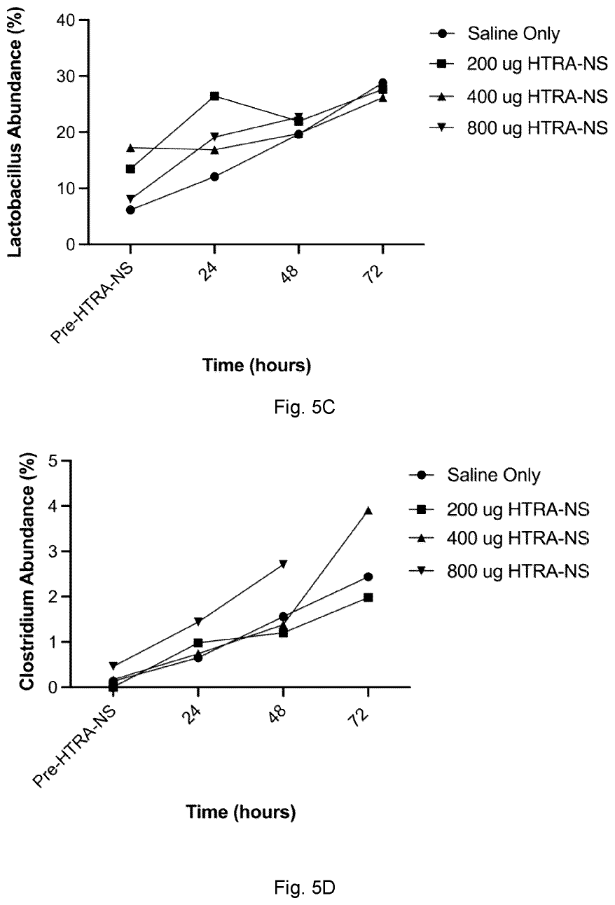

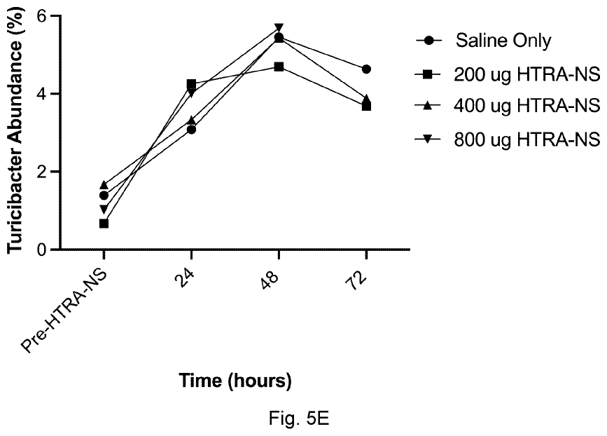

Referring to FIGS. 5A-5E, graphs show that 16S rRNA sequencing of mouse stool exposed to different concentrations of HTRA-NS (200, 400 and 800 ug) did not result in significant changes to specified predominating gut bacterial populations compared to mice given saline only (controls).

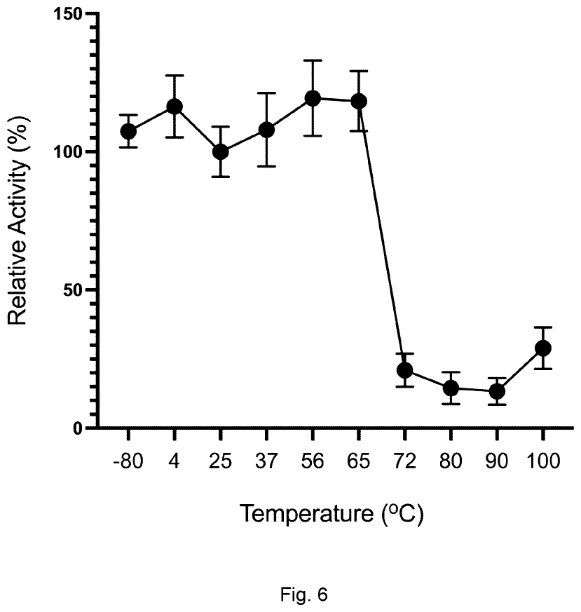

Referring to FIG. 6, a line graph shows the impact of temperature on the relative activity of HTRA. Importantly, this temperature stability data showed that HTRA-NS retained its activity up to 65° C.

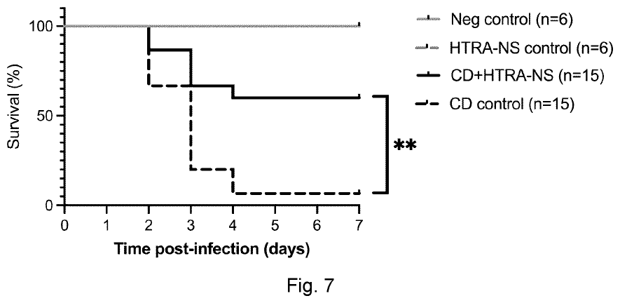

Referring to FIG. 7, a Kaplan-Meier survival curve is shown which shows the probability of survival when treated with HTRA-NS. The probability was found to be 60% whereas untreated mice had a 6.7% chance of surviving CDI in this model (p=0.003**). The survival study was conducted 3 times with 3 independent experiments. Notably, it showed a 10-fold survival benefit in mice (n=15 for each treatment group).

Referring to the Working Examples, the HTRA-1-NS) protein has been evaluated in a mouse model of C. difficile to quantify its protective capacity. The protein in this mouse model was obtained using recombinant HTRA live bacteria to produce HTRA (SEQ ID NO: 1). The HTRA was then concentrated and underwent quality control prior to use. As described herein, HTRA was found to be protective against CD illness (see FIGS. 3A-3D).

As described herein, an amount of 100-800 ug of the purified protein(s) described herein provided an effective therapy against CD and other toxin-producing bacteria. In one embodiment, the concentration was 200-800 ug of the purified protein(s) described herein. This protein therapy inactivated bacterial toxins. Although not wishing to be bound by theory, the inventors suggest that the mechanism of action of the protein therapy is unlike that used by antibiotics, which target the bacteria itself and that are susceptible to the development of resistance. Because of this difference in mechanism, the protein therapy would not be detrimentally affected by the development of resistance.

The invention also provides a combination therapy in which two or more therapeutic compounds are administered, for example HTRA protein (SEQ ID NO: 1) or HTRA-NS protein (SEQ ID NO: 3), and one or more additional therapeutic agent selected from antivirals, antibiotics, antibacterial agents, and/or antiproteases. Each of the therapeutic compounds may be administered by the same route or by a different route. Also, the compounds may be administered either at the same time (i.e., simultaneously) or each at different times. In some treatment regimens it may be beneficial to administer one of the compounds more or less frequently than the other.

Dispersions comprising the therapeutic compound(s) can be prepared in glycerol, liquid polyethylene glycols, and mixtures thereof, and in oils. Under ordinary conditions of storage and use, these preparations may contain a preservative to prevent the growth of microorganisms. Pharmaceutical compositions suitable for injectable use include sterile aqueous solutions (where water soluble) or dispersions and sterile powders for the extemporaneous preparation of sterile injectable solutions or dispersions. In all cases, the composition must be sterile and must be fluid to the extent that it can be administered easily by syringe. It must be stable under the conditions of manufacture and storage and must be preserved against the contaminating action of microorganisms such as bacteria and fungi. The vehicle can be a solvent or dispersion medium containing, for example, water, ethanol, polyol (for example, glycerol, propylene glycol, liquid polyethylene glycol, and the like), suitable mixtures thereof, and oils (e.g., vegetable oil). The proper fluidity can be maintained, for example, by the use of a coating such as lecithin, by the maintenance of the required particle size in the case of dispersion, and by the use of surfactants.

Sterile injectable solutions can be prepared by incorporating the therapeutic compound in the required amount in an appropriate solvent with one or a combination of ingredients enumerated above, as required, followed by filter sterilization. Generally, dispersions are prepared by incorporating the therapeutic compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yield a powder of the active ingredient (i.e., the therapeutic compound) optionally plus any additional desired ingredient from a previously sterile-filtered solution thereof.

Solid dosage forms for oral administration include ingestible capsules, tablets, pills, lollipops, powders, granules, elixirs, suspensions, syrups, lozenge, wafers, buccal tablets, sublingual tablets, troches, and the like. Other routes of administration include enemas or suppositories. In such solid dosage forms the active compound is mixed with at least one inert, pharmaceutically acceptable excipient or diluent or assimilable edible vehicle such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof, or incorporated directly into the subject's diet. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like. The percentage of the therapeutic compound in the compositions and preparations may, of course, be varied. The amount of the therapeutic compound in such therapeutically useful compositions is such that a suitable dosage will be obtained.

Liquid dosage forms for oral administration include pharmaceutically acceptable emulsions, solutions, suspensions, lozenge, syrups, and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, ground nut corn, germ olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

Therapeutic compounds can be administered in time-release or depot form, to obtain sustained release of the therapeutic compounds over time. The therapeutic compounds of the invention can also be administered transdermally (e.g., by providing the therapeutic compound, with a suitable vehicle, in patch form).

The following disclosure should not be construed as limiting the invention in any way. One of skill in the art will appreciate that numerous modifications, combinations, rearrangements, etc. are possible without exceeding the scope of the invention.

For example, in the descriptions of various embodiments, references to sequences and sequence listings are made. Those of ordinary skill in the art will readily appreciate that the invention is not limited to the specific sequences described, as many variants are possible without departing from the invention. For example, substitutions, mutations, deletions, and/or additions of one or more nucleotides or amino acids may be made, or may occur, without substantial effect on functional properties of embodiments described herein. Such a functional equivalent may have, for example, 60%, or 70%, or 80%, or 90%, or 95%, or 99%, or more sequence identity with a sequence described herein. Such functional equivalents are intended to be included in the embodiments of the invention.

Thus, while the invention has been described with an emphasis upon various embodiments, it will be understood by those of ordinary skill in the art that variations of the disclosed embodiments can be used, and that it is intended that the invention can be practiced otherwise than as specifically described and/or claimed herein. The invention is further illustrated by the following nonlimiting examples.

### EXAMPLE 1

### In vivo CD mouse model

Female C57B16/J mice from Jackson Laboratory (USA) were acclimated to the facility for 5 days prior to experimental use. Mice received a cocktail of antibiotics, including kanamycin 0.4 mg/ml (Sigma, Israel), gentamicin 0.035 mg/ml (Amresco, USA), colistin 850 U/ml (Sigma, China), metronidazole 0.215 mg/ml (Sigma, China) and vancomycin 0.045 mg/ml (Sigma, Israel), ad libitum in drinking water for 3 days. Mice were then infected by oral gavage with C. difficile (1×10⁵ CFU/mL of vegetative cells).

Mice were visually inspected each day for phenotypic changes associated with CDI including measurements of daily weight loss, level of activity monitored for 20 mins per day, changes in posture, fur, stool consistency, and eye appearance as parameters of illness all of which would contribute to a total clinical score and severity of CDI. Mice experiencing >10% weigh-loss post infection (p.i) or showing clinical signs consistent with CDI were sacrificed immediately. See FIG. 7 for a plot of percent survival vs. days post infection. See FIG. 3D for in vivo data on the activity of HTRA-NS from mouse stool obtained from mice infected with CD and treated with HTRA-NS.

Histology: Murine intestinal tissues, colons and ceca, were first measured to assess colonic shortening and then fixed in 10% formalin followed by 70% ethanol. Fixed tissues were processed and embedded in paraffin, and 4-μm-thick sections were stained with hematoxylin and eosin (H&E, Thermo Fisher Scientific, USA). Stained sections were examined blinded by a board-certified gastrointestinal pathologist, using an established graded scoring system. The scoring system took into consideration 1) neutrophil migration and tissue infiltration, 2) hemorrhagic congestion, and edema of the 3) mucosa and epithelial cell damage. A score between 0 and 3 was assigned for each parameter, and the overall score was the sum of all the scores. A score of 0 indicated no pathological damage, 1 mild, 2 moderate and 3 severe; the sum in scores represented the total damage in the tissue. Representative images of the tissues were recorded using a microscope (Olympus BX71, USA) with a digital camera (INFINITY2 or Qimagining Retiga-2000RV, USA).

### EXAMPLE 2

### Toxin Quantification by Western Blot and ELISA

Stool pellets were collected from each mouse each day and stored according to laboratory protocol. TcdA and TcdB levels were quantified in murine stool collected each day and from stool collected from the colons of mice after euthanization. Stool were quantified using a tgcBIOMICS ELISA kit (Bingen, Germany) as per the kit manufacturer's instructions. Briefly, 50 mg of stool from mice were resuspended in an Eppendorf tube with 450 μL of the kit dilution buffer and centrifuged at 2500 xg for 20 min. 100 μL of supernatant was added to pre-coated wells against TcdA/TcdB polyclonal antibodies and incubated for 1 h at room temperature. Plates were then washed (x3) with wash buffer (see manufacturer's instructions, and incubated with toxin specific secondary antibodies (either anti-TcdA-HRP or TcdB-HRP conjugate) for 30 minutes. Following washes with wash buffer (x3), 100 μL of the substrate was added to each well and incubated for 15 mins at room temperature. 50 μL of stop solution (see manufacturer's instructions) was added to each well and absorbance at 450 and 620 nm were measured through a microplate reader (Bio-Tek uQuant MQX200). A standard curve was generated using TcdA and TcdB pure toxin provided in the kit to calculate sample concentrations. All specimens were tested in triplicate.

Concentrated proteins were incubated with TcdA and TcdB and toxin degradation was measured using Western blots. The protein HTRA-NS (SEQ ID NO: 3) proteolyzed both TcdA and TcdB in a dose dependent manner. 30 ug of HTRA-NS proteolyzed 50 ng of TcdA and 50 ng of TcdB (see FIGS. 1A and 1B).

### EXAMPLE 3

### Toxin Activity Using a Cell Rounding Assay

In an in vitro study of toxin activity using a cell rounding assay, NIH 3T3 fibroblasts (ATCC) were proliferated and seeded in 24-well flat-bottom tissue culture plates with DMEM media (GIBCO, Thermo Fischer Scientific, USA) supplemented with 10% fetal calf serum (GIBCO, Thermo Fischer Scientific, USA) at 37° C. in 5% CO₂ and incubated for 48-72 hours. Fibroblasts were examined first at 48 hours to assess cellular confluency, a 70% confluent cellular monolayer was a pre-requisite prior to being used in cell rounding assays.

In an in vivo study of toxin activity using a cell rounding assay, the impact of CD toxin in stool was evaluated. Specifically, 50 mg of murine stool was homogenized in 500 μl of phosphate buffered saline (pH 7.2). The supernatants were recovered after 30 min of centrifugation at 16,000Xg. Fibroblasts were exposed to fresh supernatants from control and treated groups and incubated 2 h at 37° C. with 5% CO₂. After incubation, all wells were washed with phosphate buffer saline (PBS) and the cells fixed with 10% phosphate-buffered formalin (Fisher Scientific, Belgium). Following 30 min of incubation at room temperature, cells were rinsed twice with PBS and stained with Giemsa (Sigma Aldrich, USA). Stains were incubated overnight and then washed out with PBS. Cells was imaged using a microscope (Olympus BX71) at 10X with a digital camera (Qimiging, Retinga-2000RV,FAST1394). Cells were counted using Image J 1.51a software (NIH, USA). See FIG. 2A for results of in vitro neutralization capacity of HTRA against TcdA.

### EXAMPLE 4

### Protein Expression

Protein expression of genes cloned into E. coli were induced using Isopropyl-b-D-1-thiogalatopyranoside (IPTG) (Hansen, L. H., et al. Curr Microbiol 36, 341-347, (1998)) and cell pellets were harvested, sonicated, and clear supernatant was loaded onto a Ni-NTA (Qiagen) column. Each fraction was collected and absorbances was measured at 280 nm, peak fractions containing the protein were confirmed by SDS-PAGE, casein zymography gels and western blot analysis using a 6X histidine-tagged antibody. Fractions containing the protein of interest were concentrated by using a 10 kD cut-off centrifugal device (Millipore, Sigma) and buffer exchanged to 50 mM Tris, 50 mM NaCl (pH 7.5). Aliquots of each of the individual proteins were made and flash frozen with liquid nitrogen and stored at '80° C.

### EXAMPLE 5

### Assessment of in vivo Toxicity of Purified HTRA-NS

Toxicity was assessed in C57/BL6/J mice exposed and unexposed to antibiotics. Briefly, mice received either no antibiotics, or a cocktail of antibiotics, including kanamycin 0.4 mg/ml (Sigma, Israel), gentamicin 0.035 mg/ml (Amresco, USA), colistin 850 U/ml (Sigma, China), metronidazole 0.215 mg/ml (Sigma, China) and vancomycin 0.045 mg/ml (Sigma, Israel), ad libitum in drinking water for 3 days (Chen, X., et al. Gastroenterology 135, 1984-1992, (2008)). Mice were gavaged with either 0.9% saline, 200 ug, 400 ug or 800 ug of purified HTRA once a day for three consecutive days (3 mice/group, 6 groups). Mice were monitored for clinical signs of discomfort/toxicity and sacrificed at day-3 post HTRA/saline gavage. The impact of HTRA-NS was assessed measuring changes in body weight (FIG. 4A) and histological changes including colonic shortening in mice (FIG. 4B). Changes in the stool microbiota were measured by 16S rRNA sequencing of murine stool bacteria over time (FIG. 4C).

### EXAMPLE 6

### In vivo Studies of HTRA-NS

The efficacy of protection afforded HTRA-NS was evaluated in vivo. The antibiotic CDI mouse model that has been used previously was used to determine the efficacy of protection of HTRA in vivo (Chen, X. et al. Gastroenterology 135, 1984-1992, (2008)). Post antibiotics, mice were administered via oral gavage, with 400 ug of purified HTRA-NS protein (4 mice/group) once per day for three consecutive days. Mice rested for 24 hours and were infected by oral gavage with C. difficile (1×10⁵ CFU/mL of vegetative cells). Controls for this experiment included mice that received oral gavages with HTRA but no CD infection to show that the HTRA-NS was not leading to weight loss or death in mice (3 mice/group; negative controls), saline (3 mice/group; vehicle control), and CD infection with ribotype-027 (3 mice/group; positive controls). Positive controls were euthanized at 48 hours p.i as these mice progress rapidly to clinical disease. HTRA pre-treated animals exposed to CD were monitored for clinical disease for up to 72 hours post CD exposure provided that mice did not experience significant weight-loss (e.g., >15% or >25% of body weight). This experiment was repeated 3 independent times. At 72 hours, all mice were euthanized and intestinal tissue was processed and scored for histology. Stool pellet was assayed for TcdA and TcdB toxin concentrations and toxin activity was evaluated using a cell rounding assay.

### EXAMPLE 7

### Optimizing Dosing, Administration Frequency, and Timing of Administration of HTRA-NS (SEQ ID NO: 3) in vivo

The safety of using HTRA was established via a dosing study where different concentrations of HTRA were tested in mice exposed to antibiotics. Four different doses were evaluated. Mice received a cocktail of antibiotics in drinking water as previously described (Chen, X. et al. Gastroenterology 135, 1984-1992, (2008)). Mice were gavaged with either 0.9% saline, dose 1 (200 ug), dose 2 (400 ug), and dose 3 (800 ug) of purified HTRA-NS once a day for three consecutive days (3 mice/group, 6 groups). Mice then rested for 24 hours. Controls for this experiment included mice that received oral gavages with Tris buffer (the buffer that HTRA-NS is solubilized in) (negative controls). All mice were monitored for clinical disease and mice were euthanized after 72 hours (see FIGS. 4A-C). Analysis of tissue, systemic inflammation and the GI microbiota was performed as outlined in Example 5.

### EXAMPLE 8

### Gel Electrophoresis Studies

Purified HTRA-NS protein from Parabacteriodes distanosis degraded TcdA in vitro. Western blot assay of TcdA following incubation of HTRA-NS protein, 0.3, 1, 3, 10, 30, 100 ug, with purified TcdA toxin (50 ng) for 60 minutes at 37° C. No degradation of TcdA was observed at concentrations of HTRA-NS 0.3 to 30 ug however, at 100 ug a significant degradation of TcdA was observed. TcdA incubated at 37° C. for 60 minutes as a control did not result in toxin degradation (FIG. 1A).

Purified HTRA-NS protein from Parabacteriodes distanosis degraded TcdB in vitro. Western blot assay of TcdA following incubation of HTRA-protein, 0.3, 1, 3, 10, 30, 100 ug, with purified TcdB toxin (50 ng) for 60 minutes at 37° C. No degradation of TcdB was observed at concentrations of HTRA-NS 0.3 to 1 ug. Concentrations of 3 ug of HTRA-NS incubated with TcdB protein resulted in a 50% degradation and higher concentrations (10, 30, 100 ug) of HTRA-NS led to 95% loss of detectable TcdB protein by western blot. TcdB incubated at 37° C. for 60minutes as a control resulted in no TcdB degradation (see FIG. 1B).

### EXAMPLE 8

### Temperature Stability Studies

To evaluate the impact of temperature on protease activity of HTRA-NS, five independently generated preparations of HTRA-NS, stored at −80° C., were thawed and diluted to a concentration of 1.2 ug/ul in reaction buffer (50 mM Tris, pH 7.5, 150 mM NaCl). HTRA-NS preparations were incubated at −80, 4, 22, 37, 56, 65, 72, 80, 90, and 100° C. for 15 minutes. 60ul of the HTRA-NS reaction buffer preparations were then added to a 96-well black half area plate (CoStar), each preparation was tested individually in triplicate wells. The activity of HTRA-NS was then evaluated using a FITC-Casein Fluorescent Protease Assay Kit (ThermoFisher

Scientific). FITC-Casein was diluted in the reaction buffer to a concentration of 10 ug/mL and 60 uL of this working solution was added to each well of the plate. The plate was then covered with tin foil and left on a shaker for 30 minutes before reading (485/538 nm, Excitation/Emission) by using a Spectra Max M3 plate reader (Molecular Devices). Background Fluorescence (control well) was subtracted from all the samples and relative fluorescent units (RFUs) were obtained by normalizing to fluorescence at room temperature. See FIG. 6 for a graph of relative activity of HTRA-NS vs. temperature.

### EQUIVALENTS

It will be understood by those skilled in the art that this description is made with reference to certain embodiments and that it is possible to make other embodiments employing the principles of the invention which fall within its spirit and scope.

SEQUENCE LISTING

```
SEQ ID NO: 1 HTRA protein (507 amino acids)
Met Lys Asn Ile Trp Lys Asn Val Leu Gly Val Ala Leu Ile Ala Ala
1 5 10 15
Ile Ser Ser Gly Ala Ala Ile Gly Thr Ser Thr Tyr Leu Met Asn Lys
                        20 25 30
Asn Gln Arg Pro Ala Glu Leu Ala Ser Gly Val Glu Asn Thr Phe Lys
                35 40 45
Gln Pro Tyr Arg Leu Thr Asn Tyr Gly Thr Val Ala Ala Glu Asn Ile
        50 55 60
Asp Phe Thr Thr Ala Ala Glu Ser Ala Ile His Gly Val Val His Ile
65 70 75 80
Lys Ala Thr Ala Asn Ala Gln Ala Ser Asn Gly Asp Gly Gly Gln Gln
                                85 90 95
Tyr Met Asp Pro Phe Glu Tyr Phe Phe Gly Phe Gly Gly Arg Gly Gly
                        100 105 110
Phe Gln Arg Pro Gln Gln Gln Pro Arg Val Gly Ala Gly Ser Gly Val
                115 120 125
Ile Ile Ser Thr Asp Gly Tyr Ile Ile Thr Asn Asn His Val Ile Asp
        130 135 140
Gly Ala Asp Glu Leu Glu Val Thr Leu Asn Asp Asn Arg Lys Phe Pro
145 150 155 160
Ala Lys Ile Ile Gly Ala Asp Pro Thr Thr Asp Ile Ala Leu Ile Lys
                                165 170 175
Ile Glu Ala Thr Asp Leu Pro Thr Ile Pro Phe Gly Asp Ser Glu Lys
                        180 185 190
Leu Lys Val Gly Glu Trp Val Leu Ala Val Gly Asn Pro Phe Asn Leu
                195 200 205
Thr Ser Thr Val Thr Ala Xaa Ile Val Ser Ala Lys Ser Arg Gly Asn
        210 215 220
Ile Gly Ala Gly Gly Lys Asp Arg Ser Lys Ile Glu Ser Phe Ile Gln
225 230 235 240
Thr Asp Ala Ala Val Asn Pro Gly Asn Ser Gly Gly Ala Leu Val Asn
                                245 250 255
Thr Lys Gly Glu Leu Val Gly Ile Asn Thr Ala Ile Tyr Ser Glu Thr
                        260 265 270
Gly Asn Phe Ala Gly Tyr Ser Phe Ala Val Pro Ile Ser Ile Ala Gly
                275 280 285
Lys Val Ala Asn Asp Leu Lys Gln Phe Gly Thr Val Gln Arg Ala Val
        290 295 300
Leu Gly Val Leu Ile Gln Asp Pro Gln Tyr Val Pro Asp Ala Glu Lys
305 310 315 320
Glu Lys Val Lys Val Phe Glu Gly Ala Tyr Val Gly Gly Phe Ala Glu
                                325 330 335
Arg Ser Ser Ala Lys Glu Ala Gly Ile Glu Lys Gly Asp Val Ile Val
                        340 345 350
Ala Val Asn Gly Val Lys Ile Lys Ser Ser Ser Ala Leu Gln Glu Gln
                355 360 365
Ile Ser Lys Tyr Arg Pro Gly Asp Lys Val Glu Leu Thr Ile Asn Arg
        370 375 380
Asn Gly Ser Thr Lys Lys Phe Thr Val Glu Leu Arg Asn Ala Gln Gly
385 390 395 400
Ser Thr Lys Val Val Lys Gly Gly Asp Ser Ala Glu Val Met Gly Ala
                                405 410 415
Ala Phe Lys Ala Leu Asn Asp Glu Gln Lys Arg Lys Leu Gly Val Ser
                        420 425 430
Tyr Gly Ile Glu Val Thr Gly Leu Thr Ser Gly Lys Leu Lys Asp Ala
                435 440 445
Gly Ile Lys Lys Gly Phe Ile Ile Met Ile Val Asn Asn Gln Lys Ile
        450 455 460
Ser Ala Pro Glu Asp Leu Glu Lys Ile Val Glu Ser Ile Leu Gln Gly
465 470 475 480
Arg Thr Glu Asp Gln Gly Leu Phe Ile Lys Gly Phe Tyr Pro Asn Gly
                                485 490 495
Arg Thr Lys Tyr Tyr Ala Ile Asp Leu Ala Glu
                        500 505
SEQ ID NO: 2 HTRA genetic sequence (1521 nucleotides)
```

| | |
|---|---|
| atgaaaaata tctggaagaa tgtgcttggt gttgctctga tagcagccat tagttctggt | 60 |
| gccgcgatcg gaacgagtac ttacctgatg aataagaatc aacgtccggc ggagttggcc | 120 |
| tccggagttg agaatacctt caagcaaccg taccgattga cgaattatgg aaccgtggcc | 180 |
| gccgagaata tcgatttcac tacggccgcc gagagcgcca tccacggggt tgtccatatc | 240 |
| aaggccacgg ctaatgcgca agcctctaac ggggacggcg gtcagcagta tatggacccg | 300 |
| ttcgagtatt tcttcggctt cggtggccgt ggagggttcc aacgtccgca acagcaacct | 360 |
| cgtgtgggcg ccggctcggg tgtcattatc tctacggatg gttatatcat cacgaacaac | 420 |
| cacgtgatcg acggagcaga tgagctggag gttaccttga acgataaccg gaaattcccg | 480 |
| gcaaagatta taggggctga tccgacaaca gatatcgcct tgatcaagat cgaggcaacc | 540 |
| gacttaccta cgatcccctt tggtgattct gagaagttga aagtaggtga gtgggtgttg | 600 |
| gcggtaggta acccgtttaa cttgacctct accgttacgg caggkatcgt gagcgctaag | 660 |
| agccgtggta acatcggcgc cggtggcaag gatcgtagca agatcgagtc gtttatacag | 720 |
| acagacgccg ccgtgaaccc gggtaacagt ggtggggcgc tagtgaacac gaaaggcgag | 780 |
| ttagtcggta tcaataccgc tatctactcc gagaccggta acttcgccgg atactctttc | 840 |
| gccgtgccta tcagcatcgc cggtaaggtg gctaacgact tgaagcaatt tggaaccgtg | 900 |
| cagcgtgctg tattaggcgt attgatacaa gacccgcaat acgtgcccga cgctgagaaa | 960 |
| gagaaagtga aagtattcga gggcgcttac gtaggtggtt tcgccgagcg cagctccgct | 1020 |
| aaggaagccg gaatcgagaa gggtgacgtg attgtagctg tgaacggcgt gaagatcaaa | 1080 |
| tcgtctagcg ctttgcaaga gcagatcage aaataccgtc cgggcgacaa ggtagagttg | 1140 |
| acgatcaacc gcaacggtag cacgaagaaa ttcacggtag aactccgtaa tgcgcaaggt | 1200 |
| agcaccaagg tcgtgaaggg tggcgacagc gcagaggtta tgggcgcagc gttcaaggct | 1260 |
| ttgaacgacg agcagaaacg taagttaggt gtaagttatg gtatcgaggt caccggtctg | 1320 |
| accagcggca aactgaaaga cgccggtata aagaaaggct tcattatcat gattgtaaac | 1380 |
| aaccagaaga tctctgctcc ggaagacttg gaaaagattg tggaaagcat acttcaagga | 1440 |
| cgtacggaag atcaaggcct cttcattaaa ggcttctacc cgaacggacg tacgaagtac | 1500 |
| tatgcgatag atcttgccga a | 1521 |

```
SEQ ID NO: 3 HTRA-NS protein (484 amino acids)
Met Thr Ser Thr Tyr Leu Met Asn Lys Asn Gln Arg Pro Ala Glu Leu
1 5 10 15
Ala Ser Gly Val Glu Asn Thr Phe Lys Gln Pro Tyr Arg Leu Thr Asn
                        20 25 30
Tyr Gly Thr Val Ala Ala Glu Asn Ile Asp Phe Thr Thr Ala Ala Glu
                35 40 45
Ser Ala Ile His Gly Val Val His Ile Lys Ala Thr Ala Asn Ala Gln
        50 55 60
Ala Ser Asn Gly Asp Gly Gly Gln Gln Tyr Met Asp Pro Phe Glu Tyr
65 70 75 80
Phe Phe Gly Phe Gly Gly Arg Gly Gly Phe Gln Arg Pro Gln Gln Gln
                                85 90 95
Pro Arg Val Gly Ala Gly Ser Gly Val Ile Ile Ser Thr Asp Gly Tyr
                        100 105 110
Ile Ile Thr Asn Asn His Val Ile Asp Gly Ala Asp Glu Leu Glu Val
                115 120 125
Thr Leu Asn Asp Asn Arg Lys Phe Pro Ala Lys Ile Ile Gly Ala Asp
        130 135 140
Pro Thr Thr Asp Ile Ala Leu Ile Lys Ile Glu Ala Thr Asp Leu Pro
145 150 155 160
Thr Ile Pro Phe Gly Asp Ser Glu Lys Leu Lys Val Gly Glu Trp Val
                                165 170 175
Leu Ala Val Gly Asn Pro Phe Asn Leu Thr Ser Thr Val Thr Ala Xaa
                        180 185 190
Ile Val Ser Ala Lys Ser Arg Gly Asn Ile Gly Ala Gly Gly Lys Asp
                195 200 205
Arg Ser Lys Ile Glu Ser Phe Ile Gln Thr Asp Ala Ala Val Asn Pro
        210 215 220
Gly Asn Ser Gly Gly Ala Leu Val Asn Thr Lys Gly Glu Leu Val Gly
225 230 235 240
Ile Asn Thr Ala Ile Tyr Ser Glu Thr Gly Asn Phe Ala Gly Tyr Ser
                                245 250 255
Phe Ala Val Pro Ile Ser Ile Ala Gly Lys Val Ala Asn Asp Leu Lys
                        260 265 270
Gln Phe Gly Thr Val Gln Arg Ala Val Leu Gly Val Leu Ile Gln Asp
                275 280 285
Pro Gln Tyr Val Pro Asp Ala Glu Lys Glu Lys Val Lys Val Phe Glu
        290 295 300
Gly Ala Tyr Val Gly Gly Phe Ala Glu Arg Ser Ser Ala Lys Glu Ala
305 310 315 320
Gly Ile Glu Lys Gly Asp Val Ile Val Ala Val Asn Gly Val Lys Ile
                                325 330 335
Lys Ser Ser Ser Ala Leu Gln Glu Gln Ile Ser Lys Tyr Arg Pro Gly
                        340 345 350
Asp Lys Val Glu Leu Thr Ile Asn Arg Asn Gly Ser Thr Lys Lys Phe
                355 360 365
Thr Val Glu Leu Arg Asn Ala Gln Gly Ser Thr Lys Val Val Lys Gly
        370 375 380
Gly Asp Ser Ala Glu Val Met Gly Ala Ala Phe Lys Ala Leu Asn Asp
385 390 395 400
Glu Gln Lys Arg Lys Leu Gly Val Ser Tyr Gly Ile Glu Val Thr Gly
                                405 410 415
Leu Thr Ser Gly Lys Leu Lys Asp Ala Gly Ile Lys Lys Gly Phe Ile
                        420 425 430
Ile Met Ile Val Asn Asn Gln Lys Ile Ser Ala Pro Glu Asp Leu Glu
                435 440 445
Lys Ile Val Glu Ser Ile Leu Gln Gly Arg Thr Glu Asp Gln Gly Leu
        450 455 460
Phe Ile Lys Gly Phe Tyr Pro Asn Gly Arg Thr Lys Tyr Tyr Ala Ile
465 470 475 480
Asp Leu Ala Glu
SEQ ID NO: 4 HTRA-NS genetic sequence (1452 nucleotides)
```

| | |
|---|---|
| atgacgagta cttacctgat gaataagaat caacgtccgg cggagttggc ctccggagtt | 60 |
| gagaatacct tcaagcaacc gtaccgattg acgaattatg gaaccgtggc cgccgagaat | 120 |
| atcgatttca ctacggccgc cgagagcgcc atccacgggg ttgtccatat caaggccacg | 180 |
| gctaatgcgc aagcctctaa cggggacggc ggtcagcagt atatggaccc gttcgagtat | 240 |
| ttcttcggct tcggtggccg tggagggttc caacgtccgc aacagcaacc tcgtgtgggc | 300 |
| gccggctcgg gtgtcattat ctctacggat ggttatatca tcacgaacaa ccacgtgatc | 360 |
| gacggagcag atgagctgga ggttaccttg aacgataacc ggaaattccc ggcaaagatt | 420 |
| ataggggctg atccgacaac agatatcgcc ttgatcaaga tcgaggcaac cgacttacct | 480 |
| acgatcccct ttggtgattc tgagaagttg aaagtaggtg agtgggtgtt ggcggtaggt | 540 |
| aacccgttta acttgacctc taccgttacg gcaggkatcg tgagcgctaa gagccgtggt | 600 |
| aacatcggcg ccggtggcaa ggatcgtagc aagatcgagt cgtttataca gacagacgcc | 660 |
| gccgtgaacc cgggtaacag tggtggggcg ctagtgaaca cgaaaggcga gttagtcggt | 720 |
| atcaataccg ctatctactc cgagaccggt aacttcgccg gatactcttt cgccgtgcct | 780 |
| atcagcatcg ccggtaaggt ggctaacgac ttgaagcaat ttggaaccgt gcagcgtgct | 840 |
| gtattaggcg tattgataca agacccgcaa tacgtgcccg acgctgagaa agagaaagtg | 900 |
| aaagtattcg agggcgctta cgtaggtggt ttcgccgagc gcagctccgc taaggaagcc | 960 |
| ggaatcgaga agggtgacgt gattgtagct gtgaacggcg tgaagatcaa atcgtctagc | 1020 |
| gctttgcaag agcagatcag caaataccgt ccgggcgaca aggtagagtt gacgatcaac | 1080 |
| cgcaacggta gcacgaagaa attcacggta gaactccgta atgcgcaagg tagcaccaag | 1140 |
| gtcgtgaagg gtggcgacag cgcagaggtt atgggcgcag cgttcaaggc tttgaacgac | 1200 |
| gagcagaaac gtaagttagg tgtaagttat ggtatcgagg tcaccggtct gaccagcggc | 1260 |
| aaactgaaag acgccggtat aaagaaaggc ttcattatca tgattgtaaa caaccagaag | 1320 |
| atctctgctc cggaagactt ggaaaagatt gtggaaagca tacttcaagg acgtacggaa | 1380 |
| gatcaaggcc tcttcattaa aggcttctac ccgaacggac gtacgaagta ctatgcgata | 1440 |
| gatcttgccg aa | 1452 |

1. A method for treatment of one or more toxin(s) in a subject, the method comprising administering to the subject a therapeutically effective amount of a protein that comprises HTRA-NS protein (SEQ ID NO:3).

2. The method of claim 1, wherein the one or more toxin(s) are from one or more pathogen(s).

3. The method of claim 1 or 2, wherein the one or more pathogen(s) is Clostridioides difficile.

4. The method of any one of claims 1 to 3, wherein the subject is being administered antibiotics.

5. The method of any one of claims 1 to 4, wherein the one or more toxin(s) are selected from Toxin Clostridioides difficile A (TcdA), Toxin Clostridioides difficile B (TcdB), or Toxin Clostridioides difficile binary (CDT).

6. A method for preventing disease progression in a subject infected by Clostridioides difficile, diarrhea, or infectious colitis, the method comprising administering to the subject a therapeutically effective amount of a protein comprising HTRA-NS protein (SEQ ID NO: 3).

7. The method of claim 6, wherein the one or more toxin(s) are from one or more pathogen(s).

8. The method of claim 6 or 7, wherein the one or more pathogen(s) is Clostridioides difficile.

9. The method of any one of claims 7 to 8, wherein the subject is being administered antibiotics.

10. The method of any one of claims 6 to 9, wherein the one or more toxin(s) are selected from TcdA, TcdB, or CDT.

11. A method for the prophylaxis of one or more toxin(s) in a subject, the method comprising administering to the subject a therapeutically effective amount of a protein comprising HTRA-NS protein (SEQ ID NO: 3).

12. The method of claim 11, wherein the one or more toxin(s) are from one or more pathogen(s).

13. The method of claim 11 or 12, wherein the one or more pathogen(s) is Clostridioides difficile.

14. The method of any one of claims 11 to 13, wherein the subject is being administered antibiotics.

15. The method of any one of claims 11 to 14, wherein the one or more toxin(s) are selected from TcdA and TcdB.

16. The method of any one of claims 1 to 15, wherein the subject is a mammal.

17. The method of claim 16, wherein the mammal is a human.

18. The method of claim 16, wherein the mammal is a swine, cattle, horse, or dog.

19. A pharmaceutical preparation comprising a therapeutically effective amount of a protein comprising HTRA-NS protein (SEQ ID NO: 3) and a pharmaceutically acceptable excipient.

20. The pharmaceutical preparation of claim 19 for treatment of one or more toxin(s) in a subject.

21. The pharmaceutical preparation of claim 19 or 20, wherein the one or more toxin(s) are from one or more pathogen(s).

22. The pharmaceutical preparation of claim 21, wherein the one or more pathogen(s) is Clostridioides difficile.

23. The pharmaceutical preparation of any one of claims 21 to 22, wherein the one or more toxin(s) are selected from TcdA, TcdB, or CDT.

24. The pharmaceutical preparation of any one of claims 20 to 23, wherein the preparation is in the form of a tablet, lyophilized preparation, solution, syrup, lozenge, suppository, enema, food additive, capsule, spray, gel, cream, lotion, ointment, or foam.

25. A composition comprising a protein comprising HTRA-NS (SEQ ID NO: 3), and a pharmaceutically acceptable excipient.

26. The composition of claim 25 for treatment of one or more toxin(s).

27. The composition of claim 25 or 26, further comprising one or more additional therapeutic agents.

28. The composition of claim 27, wherein the one or more additional therapeutic agent(s) is selected from the group consisting of antivirals, antibiotics, antibacterial agents and antiproteases.

29. A protein comprising HTRA-NS (SEQ ID NO: 3).

30. The protein of claim 29 wherein the protein is a recombinant protein.

31. A nucleic acid encoding the protein of claim 29 (SEQ ID NO: 4).

32. A pharmaceutical composition comprising the nucleic acid of claim 31.

33. A composition comprising the protein of claim 29 or 30.

33. A method comprising administering the composition of claim 33.

34. The method of claim 1 wherein the treatment is prophylactic treatment.
